(19) United States
(12) Patent Application Publication
(10) Pub. No.: US 2025/0305922 A1
(43) Pub. Date: Oct. 2, 2025

(54) CONCENTRATION MEASUREMENT DEVICE, CONCENTRATION MEASUREMENT METHOD, STARTING MATERIAL VAPORIZATION SYSTEM, AND CONCENTRATION MEASUREMENT METHOD FOR STARTING MATERIAL VAPORIZATION SYSTEM

(71) Applicant: HORIBA STEC, Co., Ltd., Kyoto (JP)

(72) Inventors: Michie IMANISHI, Kyoto (JP); Toru SHIMIZU, Kyoto (JP)

(73) Assignee: HORIBA STEC, Co., Ltd., Kyoto (JP)

(21) Appl. No.: 18/868,515

(22) PCT Filed: Jan. 23, 2023
(86) PCT No.: PCT/JP2023/001886

§ 371 Date: Nov. 22, 2024

(30) Foreign Application Priority Data
Jun. 7, 2022 (JP) 2022-092083

(51) Int. Cl. G01N 7/14; C23C 16/448; G01L 9/00; G01L 11/02; G01N 33/00

(52) U.S. Cl. CPC G01N 7/14; C23C 16/448; G01L 9/0072; G01L 11/02; G01N 33/0062

(57) ABSTRACT

A concentration measurement device measures a concentration of starting material gas contained in mixed gas, and includes: a partial pressure sensor configured to detect a partial pressure of the starting material gas contained in the mixed gas, and output a partial pressure signal indicating the partial pressure; a total pressure sensor configured to detect a total pressure that is a pressure of the mixed gas, and output a total pressure signal indicating the total pressure; a delay filter configured to perform processing of delaying a response speed of the total pressure signal, and output a delayed total pressure signal that is a total pressure signal after the processing; and a calculation unit configured to calculate a concentration of the starting material gas contained in the mixed gas based on the partial pressure signal and the delayed total pressure signal.

### TECHNICAL FIELD

The present invention relates to a concentration measurement device, a concentration measurement method, a starting material vaporization system, and a concentration measurement method for the starting material vaporization system.

### BACKGROUND ART

Conventionally, as a concentration measurement device that measures a concentration of a measurement target component contained in gas, there has been provided a concentration measurement device that calculates the concentration based on a partial pressure of the measurement target component contained in the gas and a total pressure of the gas.

For example, Patent Literature 1 discloses a concentration measurement device in a starting material vaporization system. Here, the starting material vaporization system refers to a system of introducing carrier gas into a liquid or solid starting material stored in a tank to cause vaporization, and supplying mixed gas containing the carrier gas and starting material gas generated by the vaporization.

The concentration measurement device in this type of starting material vaporization system measures a concentration of the starting material gas by calculating a ratio of a total pressure that is a pressure in the tank to a partial pressure that is a pressure of the starting material gas. In this concentration measurement device, conventionally, for example, a non-dispersive infrared absorption type sensor (hereinafter, referred to as “NDIR sensor”) is used as a partial pressure sensor that measures the partial pressure, and for example, a capacitive diaphragm vacuum gauge (hereinafter referred to as “CDG”) is used as a total pressure sensor that measures the total pressure.

Here, in general, in order to obtain a sufficient SN ratio, the partial pressure sensor often performs processing such as moving average on a detected partial pressure, and outputs a partial pressure signal indicating the partial pressure. In addition, since the NDIR sensor often used as the partial pressure sensor performs processing such as chopping, a response speed of the partial pressure signal output from the NDIR is limited by a chopping frequency. As a result, the partial pressure signal output from the partial pressure sensor changes with a delay from a pressure change of the concentration measurement device.

Whereas, in general, since the total pressure sensor has a sufficiently better SN ratio than the partial pressure sensor, it is common that processing for obtaining a sufficient SN ratio, such as moving average, is not performed. Even if the processing is performed, only processing with a sufficiently faster response than the partial pressure sensor is performed. Therefore, since the total pressure sensor does not perform processing such as moving average, for example, a total pressure signal output from the total pressure sensor changes without delay from a pressure change of the concentration measurement device, as compared with the partial pressure signal output from the partial pressure sensor. As a result, a difference in response speed occurs between the partial pressure signal output from the partial pressure sensor and the total pressure signal output from the total pressure sensor.

Then, examples of a case where the pressure in the concentration measurement device changes include rising and falling of a pressure. Specifically, on an upstream side of the concentration measurement device, for example, rising of the pressure in the concentration measurement device occurs at a start of supply of the carrier gas, and for example, falling of the pressure in the concentration measurement device occurs at a stop of the supply of the carrier gas. In addition, for example, rising of the pressure in the concentration measurement device occurs when a chamber provided on a downstream side of the concentration measurement device is opened to atmosphere, and for example, falling of the pressure in the concentration measurement device occurs when the chamber is evacuated. When a pressure change such as rising or falling of the pressure in the concentration measurement device occurs, the partial pressure signal changes with a delay from the pressure change as compared with the total pressure signal. Therefore, since a difference occurs between the response speed of the partial pressure signal and the response speed of the total pressure signal, a concentration of the starting material gas cannot be accurately measured at a time of a pressure change such as rising and falling of the pressure.

### CITATION LIST

### Patent Literature

Patent Literature 1: JP 2022-38365 A

### SUMMARY OF INVENTION

### Technical Problem

Therefore, the present invention has been made to solve the above problem, and a main object thereof is to accurately measure a concentration of a measurement target component contained in gas at a time of a pressure change such as rising or falling of a pressure in a concentration measurement device.

### Solution to Problem

That is, a concentration measurement device according to the present invention is a concentration measurement device that measures a concentration of a measurement target component contained in gas, and the concentration measurement device includes: a partial pressure sensor configured to detect a partial pressure of the measurement target component contained in the gas, and output a partial pressure signal indicating the partial pressure; a total pressure sensor configured to detect a total pressure that is a pressure of the gas, and output a total pressure signal indicating the total pressure; a delay filter configured to perform processing of delaying a response speed of the total pressure signal, and output a delayed total pressure signal that is a total pressure signal after the processing; and a calculation unit configured to calculate a concentration of a component contained in the gas based on the partial pressure signal and the delayed total pressure signal.

With such a configuration, the delay filter performs processing of delaying a response speed of the total pressure signal and outputs a delayed total pressure signal which is a total pressure signal after the processing, and thus, it is possible to reduce a difference in response speed between the partial pressure signal and the delayed total pressure signal. As a result, the concentration of the measurement target component contained in the gas can be accurately measured at a time of a pressure change such as rising or falling of the pressure in the concentration measurement device.

Here, the delay filter desirably performs moving average on the total pressure signal.

In such a case, since the delay filter performs moving average on the total pressure signal, noise of the total pressure signal can be removed.

Further, it is preferable that the partial pressure signal is output by performing moving average on a detected partial pressure, and the delay filter is set such that a moving average section of the total pressure signal is included in a range of −37.5% or more and 37.5% or less with respect to a moving average section of a detected partial pressure.

In such a case, a value obtained by performing moving average on both the delayed total pressure signal and the partial pressure signal is output, and the moving average section of the total pressure signal is set in a range of −37.5% or more and 37.5% or less with respect to the moving average section of the detected partial pressure. Therefore, since a difference between the response speed of the partial pressure signal and the response speed of the delayed total pressure signal is further reduced, the concentration of the measurement target component can be measured more accurately.

In addition, an NDIR sensor may be used as the partial pressure sensor.

In the case of the partial pressure sensor using the NDIR sensor, a substance that absorbs infrared rays can be measured with high sensitivity, and the partial pressure can be measured by a measuring instrument having a simple structure and an inexpensive price.

Further, the CDG may be used as the total pressure sensor.

The total pressure sensor using the CDG can measure the total pressure with high sensitivity even when a value of the total pressure is small. However, since the CDG has a higher response speed than the NDIR sensor and hardly requires processing such as moving average for improving an SN ratio, a difference in response speed occurs between the total pressure signal and the partial pressure signal. As a countermeasure, the delay filter performs processing of delaying the response speed of the total pressure signal output from the CDG, whereby the difference in the response speed can be reduced.

The concentration measurement device desirably further includes a display control unit that outputs a concentration calculated by the calculation unit.

In such a case, the display control unit outputs a calculated concentration, so that a user can check whether the concentration measurement device is accurately performing the concentration measurement.

Further, examples of a starting material vaporization system include a starting material vaporization system that introduces carrier gas into a liquid or solid starting material stored in a tank to cause vaporization, and supplies mixed gas containing the carrier gas and starting material gas generated by the vaporization. The starting material vaporization system uses the concentration measurement device including: a partial pressure sensor configured to detect a partial pressure that is a pressure of the starting material gas, and output a partial pressure signal indicating the partial pressure; a total pressure sensor configured to detect a total pressure that is a pressure in the tank, and output a total pressure signal indicating the total pressure; a delay filter configured to perform processing of delaying a response speed of the total pressure signal, and output a delayed total pressure signal that is a total pressure signal after the processing; and a calculation unit configured to calculate a concentration of the starting material gas based on the partial pressure signal and the delayed total pressure signal.

In such a starting material vaporization system, since the delay filter outputs the delayed total pressure signal, a difference between a response speed of the partial pressure signal and a response speed of the delayed total pressure signal can be reduced. Therefore, the concentration of the starting material gas can be accurately measured at a time of a pressure change such as rising or falling of the pressure in the concentration measurement device. As a result, feedback control performed based on a measured concentration of the starting material gas can be stably performed, and thus the concentration of the starting material gas in the starting material vaporization system can be stably controlled.

Then, an example of a concentration measurement method is to measure a concentration of a component contained in gas, and the concentration measurement method includes: detecting a partial pressure of a component contained in the gas, and outputting the partial pressure as a partial pressure signal; detecting a total pressure that is a pressure of the gas, and outputting the total pressure as a total pressure signal; performing processing of delaying a response speed of the total pressure signal, and outputting a delayed total pressure signal that is a total pressure signal after the processing; and calculating a concentration of a component contained in the gas based on the partial pressure signal and the delayed total pressure signal.

According to such a concentration measurement method, processing of delaying a response speed of the total pressure signal is performed, and the delayed total pressure signal which is a total pressure signal after the processing is output, so that a difference between a response speed of the partial pressure signal and a response speed of the delayed total pressure signal can be reduced. As a result, the concentration of the component contained in the gas can be more accurately measured at a time of a pressure change.

Moreover, the concentration measurement method is desirably used in a starting material vaporization system that introduces carrier gas into a liquid or solid starting material stored in a tank to cause vaporization, and supplies mixed gas containing the carrier gas and starting material gas generated by the vaporization, and the concentration measurement method desirably measures a concentration of the starting material gas.

According to such a concentration measurement method, processing of delaying a response speed of the total pressure signal is performed, and the delayed total pressure signal which is a total pressure signal after the processing is output, so that a difference between a response speed of the partial pressure signal and a response speed of the delayed total pressure signal can be reduced. As a result, in the starting material vaporization system, the concentration of the starting material gas can be more accurately measured at a time of a pressure change such as rising or falling of the pressure in the concentration measurement device.

### Advantageous Effects of Invention

According to the present invention described above, a concentration of a measurement target component contained in gas can be accurately measured at a time of a pressure change such as rising or falling of a pressure in a concentration measurement device.

### BRIEF DESCRIPTION OF DRAWINGS

FIG. 1 is an overall schematic diagram of a starting material vaporization system according to an embodiment of the present invention.

FIG. 2 is a schematic diagram of a concentration measurement device in the embodiment.

FIG. 3 is a graph of (a) a pressure indication value and (b) a concentration calculation value in the embodiment.

FIG. 4 is a graph of (a) a pressure indication value and (b) a concentration calculation value in a conventional example.

### DESCRIPTION OF EMBODIMENT

Hereinafter, a starting material vaporization system according to an embodiment of the present invention will be described with reference to the drawings. Note that, any of the drawings shown below is schematically omitted or exaggerated as appropriate for easy understanding. The same components are denoted by the same reference numerals, and a description thereof will be omitted as appropriate.

### <1. System Configuration>

A starting material vaporization system 100 of the present embodiment is used, for example, in a semiconductor manufacturing process, and supplies, for example, starting material gas such as isopropyl alcohol (IPA) at a predetermined concentration to a drying processing chamber of a wafer cleaning device. In addition, the starting material vaporization system 100 may supply starting material gas at a predetermined concentration to a processing chamber of a semiconductor processing device such as a CVD film forming device or an MOCVD film forming device.

The starting material vaporization system 100 introduces carrier gas into a liquid or solid starting material to cause vaporization, and supplies mixed gas containing the carrier gas and starting material gas generated by the vaporization. Note that, although an example using a liquid starting material will be described below, this similarly applies to a case of using a solid starting material.

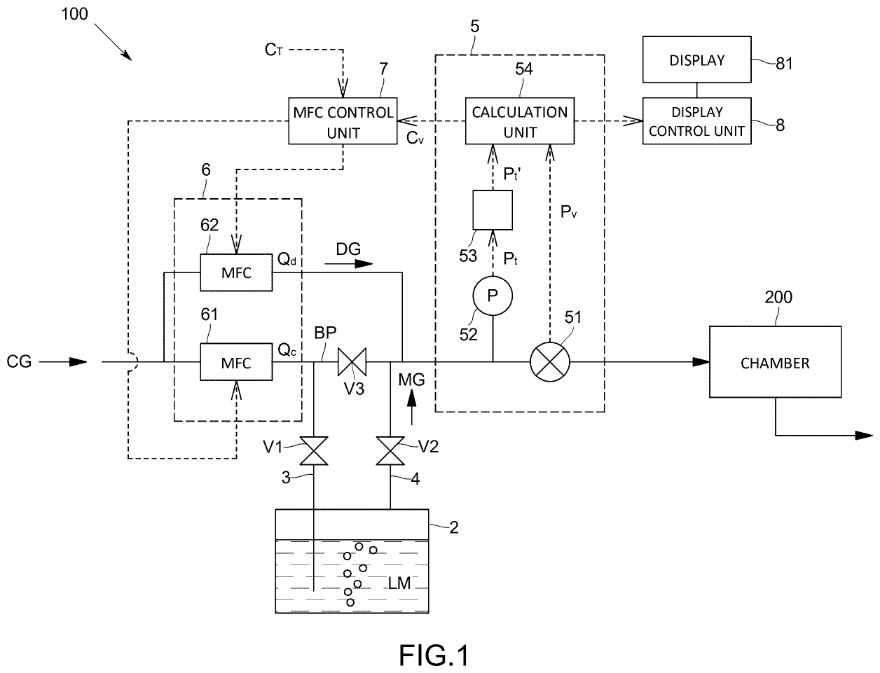

Specifically, as illustrated in FIG. 1, the starting material vaporization system 100 includes: a tank 2 that stores a liquid starting material LM; an introduction pipe 3 that introduces carrier gas CG into the tank 2 to cause bubbling; a lead-out pipe 4 that leads out mixed gas MG containing the carrier gas CG and starting material gas obtained by vaporization of the starting material LM from the tank 2; and a concentration measurement device 5 that measures a concentration of the starting material gas contained in the mixed gas MG.

The tank 2 is, for example, a stainless steel sealed container that stores the liquid starting material LM, and is heated to a constant temperature by a heating mechanism such as a heater provided outside.

A supply source (not illustrated) of the carrier gas such as nitrogen or hydrogen is connected to an upstream side of the introduction pipe 3. Further, a downstream side of the introduction pipe 3 is inserted into the tank 2. A downstream opening of the introduction pipe 3 is provided at a position lower than a liquid level of the liquid starting material LM stored in the tank 2, and the starting material LM is bubbled by the carrier gas CG introduced from the introduction pipe 3 into the tank 2. Further, the introduction pipe 3 is provided with a mass flow controller 6 that controls a flow rate in the starting material vaporization system 100. Specifically, the mass flow controller 6 includes a CG mass flow controller 61 that controls a flow rate Qc of the carrier gas CG supplied into the tank 2, and a DG mass flow controller 62 that controls a flow rate Q_{d} of dilution gas DG for diluting the mixed gas MG.

An upstream opening of the lead-out pipe 4 is connected to an upper space (gas phase) formed in a state where the liquid starting material LM is stored in the tank 2. Further, a chamber 200 of the semiconductor processing device is connected to a downstream side of the lead-out pipe 4. Moreover, the concentration measurement device 5 that measures a concentration of the starting material gas contained in the mixed gas MG is provided on a downstream side of the lead-out pipe 4 and between the lead-out pipe 4 and the chamber 200. A concentration Cᵥ of the starting material gas measured by the concentration measurement device 5 is sent to an MFC control unit 7 and a display control unit 8. The MFC control unit 7 compares the measured concentration Cᵥ of the starting material gas with a target concentration C_{T} of the starting material gas, and controls the mass flow controller 6. The display control unit 8 outputs the measured concentration Cᵥ of the starting material gas, and outputs and displays the measured concentration Cᵥ of the starting material gas on a screen such as a display 81, for example. Note that a bypass pipe BP that bypasses the tank 2 is connected to the introduction pipe 3 and the lead-out pipe 4, and the introduction pipe 3, the lead-out pipe 4, and the bypass pipe BP are provided with flow path switching valves V1 to V3 that switch between a flow path in which the carrier gas CG passes through the tank 2 and a flow path in which the carrier gas CG passes through the bypass pipe BP.

### <2. Concentration Measurement Device>

Next, the concentration measurement device 5 of the present embodiment will be described.

As illustrated in FIG. 1, the concentration measurement device 5 includes a partial pressure sensor 51 that measures a partial pressure that is a pressure of the starting material gas, a total pressure sensor 52 that measures a total pressure that is a pressure of the mixed gas MG, a delay filter 53 that performs processing of delaying a response speed of a total pressure signal Pₜ output from the total pressure sensor 52 and outputs a delayed total pressure signal Pₜ′ that is a total pressure signal after the processing, and a calculation unit 54 that calculates the concentration Cᵥ of the starting material gas contained in the mixed gas MG.

The partial pressure sensor 51 detects a partial pressure that is a pressure of the starting material gas, and outputs a partial pressure signal Pᵥ indicating the partial pressure. Specifically, the partial pressure sensor 51 is, for example, an NDIR sensor, and performs processing such as moving average on the detected partial pressure in order to obtain a sufficient SN ratio, and outputs the partial pressure signal Pᵥ indicating the partial pressure after the processing. As a result, at a time of a pressure change such as rising or falling of a pressure in the concentration measurement device 5, for example, at a time of evacuation of the chamber 200 or a start of supply of the carrier gas, the partial pressure signal Pᵥ changes with a delay from the pressure change. Note that, in the present embodiment, an evaluation speed of the chamber 200 is, but not limited to, 7.5 kPa/sec.

The total pressure sensor 52 detects a pressure of the mixed gas MG, and outputs the total pressure signal Pₜ indicating the total pressure. Specifically, the total pressure sensor 52 is, for example, the CDG. When a pressure change such as rising or falling of the pressure in the concentration measurement device 5 occurs, the total pressure signal Pₜ changes without being delayed from the pressure change as compared with the partial pressure signal Pᵥ.

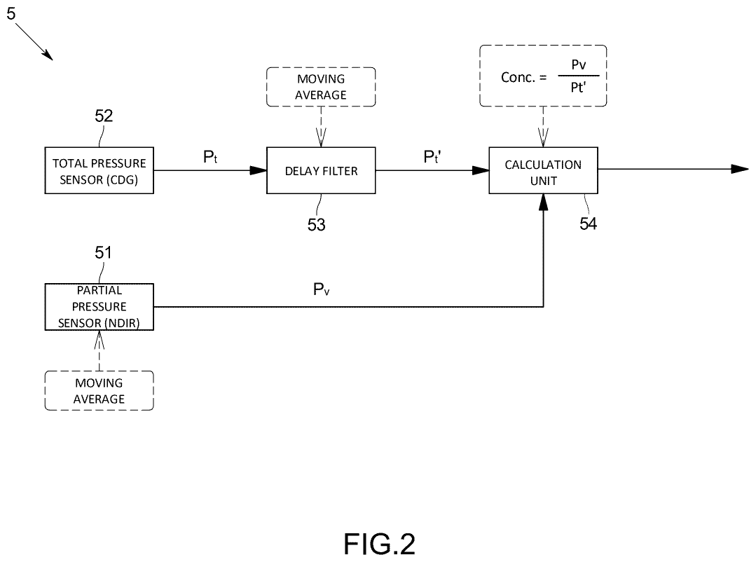

Then, the delay filter 53 continuously performs processing of delaying a response speed of the total pressure signal Pₜ output from the total pressure sensor 52, and outputs the delayed total pressure signal Pₜ′ which is a total pressure signal after the processing. Specifically, as illustrated in FIG. 2, the delay filter 53 performs processing of moving average on the total pressure signal Pₜ. Thereafter, the delay filter 53 outputs the delayed total pressure signal Pₜ′, which is a total pressure signal after the processing, to the calculation unit 54. Note that, at a time of a pressure change of the pressure in the concentration measurement device 5, such as rising or falling, the delayed total pressure signal Pₜ′ changes with a delay from the pressure change, as compared with the total pressure signal Pₜ.

The calculation unit 54 calculates the concentration Cᵥ of the starting material gas contained in the mixed gas MG, based on the partial pressure signal Pᵥ and the delayed total pressure signal Pₜ′. Specifically, as illustrated in FIGS. 1 and 2, the calculation unit 54 calculates the concentration Cᵥ of the starting material gas, which is a ratio of the partial pressure signal Pᵥ to the delayed total pressure signal Pₜ′. Thereafter, the calculation unit 54 outputs the calculated concentration Cᵥ of the starting material gas to the MFC control unit 7 and the display control unit 8.

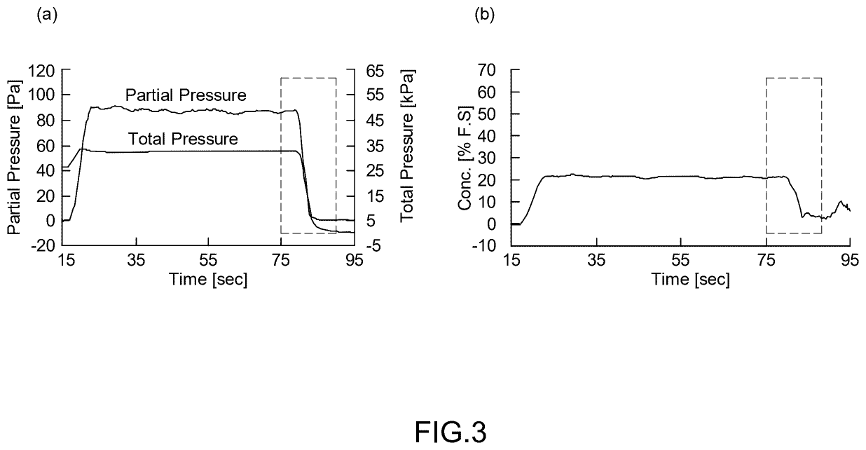

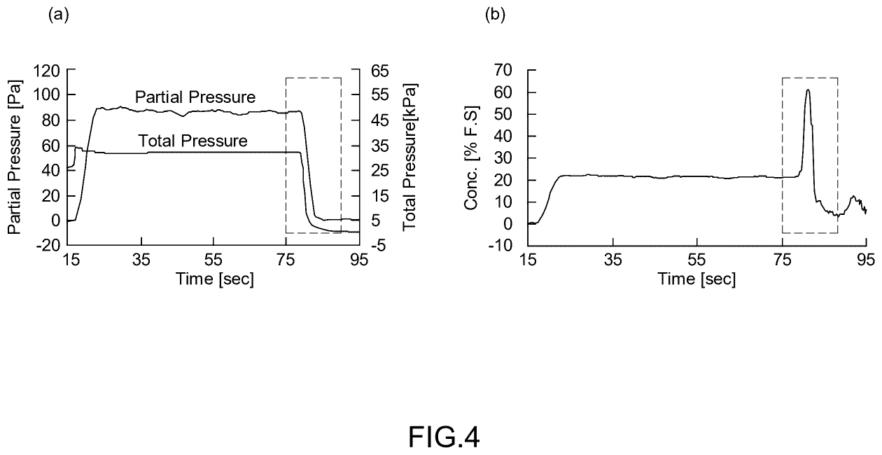

Next, a comparison between the concentration measurement device 5 in the present embodiment and a concentration measurement device of a conventional example will be described with reference to FIGS. 3 and 4.

In the concentration measurement device 5 according to the present embodiment, the delay filter 53 outputs the delayed total pressure signal Pₜ′ subjected to processing of delaying a response speed of the total pressure signal Pₜ. The delay filter 53 is set such that a moving average section of the total pressure signal Pₜ is included in a range of −37.5% or more and 37.5% or less with respect to a moving average section of the detected partial pressure. That is, when the moving average section of the detected partial pressure is set to, for example, 3.2 seconds, the moving average section of the total pressure signal Pₜ is set to be included in a range of ±1.2 seconds of the moving average section of the detected partial pressure. More preferably, the moving average section of the total pressure signal Pₜ is included in a range of −25% or more and 25% or less of the moving average section of the detected partial pressure. That is, when the moving average section of the detected partial pressure is set to 3.2 seconds, the moving average section of the total pressure signal Pₜ is desirably set to be included in a range of ±0.8 seconds of the moving average section of the detected partial pressure. In particular, in the present embodiment, the moving average section of the total pressure signal Pₜ coincides with the moving average section of the detected partial pressure. As a result, a difference between the response speed of the delayed total pressure signal Pₜ′ and the response speed of the partial pressure signal Pᵥ is reduced. Therefore, as illustrated in FIG. 3, an overshoot does not occur at a time of falling of a pressure on a downstream side of the concentration measurement device 5, for example, at a time of evacuation of the chamber 200. Note that, here, the overshoot means that the measured concentration Cᵥ of the starting material gas output by the display control unit 8 rapidly changes. In addition, the rapid change in the measured concentration Cᵥ means that the apparent concentration Cᵥ of the starting material gas changes by ±10% or more within three seconds, for example, as compared with the steady concentration Cᵥ of the starting material gas, but is not limited thereto.

Whereas, since the concentration measurement device of the conventional example is not provided with the delay filter 53, as illustrated in FIG. 4, a difference between the response speed of the partial pressure signal Pᵥ and the response speed of the delayed total pressure signal Pₜ′ is larger than that of the concentration measurement device 5. As a result, as illustrated in FIG. 4, in the concentration measurement device of the conventional example, the overshoot occurs, for example, at a time of falling of the pressure on a downstream side of the concentration measurement device.

### Effects of Present Embodiment

According to the concentration measurement device 5 of the present embodiment, the delay filter 53 performs processing of delaying a response speed of the total pressure signal Pₜ, such as moving average, on the total pressure signal Pₜ, and outputs the delayed total pressure signal Pₜ′ which is a total pressure signal after the processing. Therefore, a difference between the response speed of the partial pressure signal Pᵥ and the response speed of the delayed total pressure signal Pₜ′ can be reduced. As a result, the concentration Cᵥ of the starting material gas contained in the mixed gas MG can be accurately measured at a time of a pressure change such as rising or falling of the pressure in the concentration measurement device 5.

Further, the delay filter 53 is set such that a moving average section of the total pressure signal Pₜ is included in a range of −37.5% or more and 37.5% or less with respect to a moving average section of the detected partial pressure. In particular, in the present embodiment, the moving average section of the total pressure signal Pₜ coincides with the moving average section of the detected partial pressure. Therefore, at a time of a pressure change such as rising or falling of the pressure in the concentration measurement device 5, the difference between the response speed of the delayed total pressure signal Pₜ′ and the response speed of the partial pressure signal Pᵥ is further reduced. Therefore, it is possible to prevent occurrence of the overshoot in which the concentration Cᵥ of the starting material gas output to the display control unit 8 rapidly changes, for example, at a time of falling of the pressure on a downstream side of the concentration measurement device 5 such as evacuation of the chamber 200. As a result, the concentration Cᵥ of the starting material gas contained in the mixed gas MG can be more accurately measured at a time of falling of the pressure on the downstream side of the concentration measurement device 5. Accordingly, the starting material vaporization system 100 can stably perform feedback control by the MFC control unit 7 based on the measured concentration Cᵥ of the starting material gas, so that the measured concentration Cᵥ of the starting material gas can be stably controlled.

Furthermore, the delay filter 53 in the present embodiment reduces a difference between the response speed of the partial pressure signal Pᵥ and the response speed of the delayed total pressure signal Pₜ′. Therefore, the delay filter 53 has an effect of suppressing the overshoot occurring at a time of falling of the pressure on the downstream side of the concentration measurement device 5, but the effect of the delay filter 53 is not limited thereto. Specifically, the delay filter 53 can suppress output of a value lower than an actual measured value at a time of rising of the pressure on an upstream side of the concentration measurement device 5, for example, at a start of supply of the carrier gas.

### Other Modified Embodiments

Note that the present invention is not limited to the above embodiment.

Although the delay filter 53 in the present embodiment performs processing of delaying a response speed of a total pressure signal, such as moving average on the total pressure signal Pₜ, the processing of delaying the response speed of the total pressure signal is not limited to the moving average. For example, the processing of delaying the response speed of the total pressure signal may use a Kalman filter or the like that predicts the total pressure signal Pₜ to be output in the future from the output total pressure signal Pₜ.

In addition, the delay filter 53 in the present embodiment continuously performs the processing of delaying the response speed of the total pressure signal, but may perform the processing of delaying the response speed of the total pressure signal with switching. For example, a determination unit that determines a pressure change such as rising or falling of the pressure in the concentration measurement device 5 may be further provided, and the determination unit may determine whether or not to perform the processing of delaying the response speed of the total pressure signal. Specifically, when the determination unit determines that a pressure change such as rising or falling of the pressure in the concentration measurement device 5 is occurring, the delay filter 53 performs the processing of delaying the response speed of the total pressure signal. Further, when the determination unit determines that the change described above is not occurring, the delay filter 53 does not perform the processing of delaying the response speed of the total pressure signal. As a result, the delay filter 53 can perform the processing of delaying the response speed of the total pressure signal with switching in accordance with determination of the determination unit, so that a section in which the processing of delaying the response speed of the total pressure signal is performed can be limited.

Further, in the present embodiment, the MFC control unit 7 and the display control unit 8 are not included in the concentration measurement device 5, but the MFC control unit 7 and the display control unit 8 may be included in the concentration measurement device 5.

In addition, the partial pressure sensor in the present embodiment uses the NDIR, but the partial pressure sensor may use an infrared laser absorption method instead of the NDIR, or may use a Fourier transform infrared spectroscopy (FTIR) method. Further, a wavelength of the partial pressure sensor is not limited to an infrared region.

In addition, the present invention is not limited to the above-described embodiments, and it goes without saying that various modifications can be made without departing from the spirit of the present invention.

### INDUSTRIAL APPLICABILITY

According to the present invention, a concentration of a measurement target component contained in gas can be accurately measured at a time of a pressure change such as rising or falling of a pressure in a concentration measurement device.

### REFERENCE SIGNS LIST

100 starting material vaporization system
2 tank
3 introduction pipe
4 lead-out pipe
5 concentration measurement device
51 partial pressure sensor
52 total pressure sensor
53 delay filter
54 calculation unit

1. A concentration measurement device that measures a concentration of a measurement target component contained in gas, the concentration measurement device comprising:
a partial pressure sensor configured to detect a partial pressure of the measurement target component contained in the gas, and output a partial pressure signal indicating the partial pressure;
a total pressure sensor configured to detect a total pressure that is a pressure of the gas, and output a total pressure signal indicating the total pressure;
a delay filter configured to perform processing of delaying a response speed of the total pressure signal, and output a delayed total pressure signal that is a total pressure signal after the processing; and
a calculation unit configured to calculate a concentration of the measurement target component contained in the gas based on the partial pressure signal and the delayed total pressure signal.

2. The concentration measurement device according to claim 1, wherein the delay filter performs moving average on the total pressure signal.

3. The concentration measurement device according to claim 2, wherein
the partial pressure signal is output by performing moving average on a detected partial pressure, and
the delay filter is set such that a moving average section of the total pressure signal is included in a range of −37.5% or more and 37.5% or less with respect to a moving average section of a detected partial pressure.

4. The concentration measurement device according to claim 1, wherein the partial pressure sensor uses a non-dispersive infrared absorption type sensor.

5. The concentration measurement device according to claim 1, wherein the total pressure sensor uses a capacitive diaphragm vacuum gauge.

6. The concentration measurement device according to claim 1, further comprising a display control unit configured to output a concentration calculated by the calculation unit.

7. A starting material vaporization system that introduces carrier gas into a liquid or solid starting material stored in a tank to cause vaporization, and supplies mixed gas containing the carrier gas and starting material gas generated by the vaporization, the starting material vaporization system using the concentration measurement device comprising:
a partial pressure sensor configured to detect a partial pressure that is a pressure of the starting material gas, and output a partial pressure signal indicating the partial pressure;
a total pressure sensor configured to detect a total pressure that is a pressure in the tank, and output a total pressure signal indicating the total pressure;
a delay filter configured to perform processing of delaying a response speed of the total pressure signal, and output a delayed total pressure signal that is a total pressure signal after the processing; and
a calculation unit configured to calculate a concentration of the starting material gas based on the partial pressure signal and the delayed total pressure signal.

8. A concentration measurement method for measuring a concentration of a component contained in gas, the concentration measurement method comprising:
detecting a partial pressure of a component contained in the gas, and outputting the partial pressure as a partial pressure signal;
detecting a total pressure that is a pressure of the gas, and outputting the total pressure as a total pressure signal;
performing processing of delaying a response speed of the total pressure signal, and outputting a delayed total pressure signal that is a total pressure signal after the processing; and
calculating a concentration of a component contained in the gas based on the partial pressure signal and the delayed total pressure signal.

9. The concentration measurement method according to claim 8, wherein the concentration measurement method is used in a starting material vaporization system that introduces carrier gas into a liquid or solid starting material stored in a tank to cause vaporization, and supplies mixed gas containing the carrier gas and starting material gas generated by the vaporization, and the concentration measurement method measures a concentration of the starting material gas.
